(19) United States
(12) Patent Application Publication
(10) Pub. No.: US 2024/0150698 A1
(43) Pub. Date: May 9, 2024

(54) CELL CULTURE DEVICE AND METHOD FOR PRODUCING CELL CULTURE DEVICE

(71) Applicant: Sharp Display Technology Corporation, Kameyama City (JP)

(72) Inventors: Chihiro TACHINO, Kameyama City (JP); Tomoko TERANISHI, Kameyama City (JP); Satoshi IHIDA, Kameyama City (JP); Takeshi HARA, Kameyama City (JP)

(21) Appl. No.: 18/383,626

(22) Filed: Oct. 25, 2023

(30) Foreign Application Priority Data
Nov. 4, 2022 (JP) 2022-177028

(51) Int. Cl. C12M 1/12; C12M 1/00; C12M 1/34

(52) U.S. Cl. CPC C12M 25/14; C12M 23/22; C12M 41/00

(57) ABSTRACT

A cell culture device includes a substrate, a wire disposed on a main surface of the substrate, an insulating film disposed on the main surface of the substrate and having a part that overlaps the wire, and the electrodes disposed on the insulating film and having a part that overlaps the wire. The insulating film has a contact hole at a position overlapping the wire and the electrode. A plurality of grooves are formed on at least the surface of a part of the electrode that overlaps the contact hole.

### BACKGROUND

### 1. Field

The present disclosure relates to a cell culture device and a method for producing the cell culture device.

### 2. Description of the Related Art

In the related art, extracellular potential measuring methods using, for example, microwell plates equipped with microelectrodes have been used to record various activities of biological specimens because they can measure cell activity noninvasively and in real time. For example, Japanese Unexamined Patent Application Publication No. 2019-219244 discloses a potential measuring device including: multiple readout electrodes that are arranged in an array and detect a potential at an action potential generation point generated as a result of the action of a cell; an insulating member; a reference electrode that detects a reference potential; and an amplifier that generates a potential difference between a detected potential obtained by the readout electrodes and a detected potential obtained by the reference electrode, wherein: the readout electrodes each include a covered region in which the insulating member is stacked on the corresponding readout electrode, and an open region in which the insulating member is not stacked on the corresponding readout electrode; and the readout electrodes include, in the open region, at least one high portion that is higher than a stack surface of the corresponding readout electrode on which the insulating member is stacked, and/or at least one low portion that is lower than the stack surface.

In the potential measuring device disclosed in Japanese Unexamined Patent Application Publication No. 2019-219244, the readout electrodes are connected to signal readout wires via a switch. The readout electrodes are directly connected to the switch, but a specific connection configuration is unclear. To produce the potential measuring device, the surface of the readout electrodes is oxidized and reduced by electrochemical oxidation/reduction cycling to form the high portion and the low portion. The finished readout electrodes may vary depending on the way of electrochemical oxidation/reduction cycling, and openings may be generated in the readout electrodes in some cases so that part of each readout electrode may be isolated like islands. If portions of the readout electrodes that are not connected to the switch are isolated like islands in this case, there is a problem that the measurement is failed in the island portions to reduce measurement sensitivity.

The technique disclosed in the present disclosure is accomplished in light of the above circumstances. It is desirable to avoid reducing measurement sensitivity.

### SUMMARY

According to a first aspect of the disclosure, there is provided a cell culture device including: a substrate; a wire disposed on a main surface of the substrate; an insulating film disposed on the main surface of the substrate and having a part that overlaps the wire; and an electrode disposed on the insulating film and having a part that overlaps the wire, wherein the insulating film has a contact hole at a position overlapping the wire and the electrode, and a plurality of grooves are formed on at least a surface of a part of the electrode that overlaps the contact hole.

According to a second aspect of the disclosure, there is provided the cell culture device according to the first aspect wherein the substrate and the insulating film are each made of a light-transmissive insulating material, the wire is made of a light-shielding conducting material, and the electrode is made of a light-transmissive conducting material.

According to a third aspect of the disclosure, there is provided the cell culture device according to the first aspect or the second aspect wherein the plurality of grooves have a depth smaller than a thickness of the electrode.

According to a fourth aspect of the disclosure, there is provided the cell culture device according to the first aspect or the second aspect wherein the plurality of grooves have a depth equal to a thickness of the electrode, and the electrode includes a plurality of island portions divided by the plurality of grooves.

According to a fifth aspect of the disclosure, there is provided the cell culture device according to any one of the first aspect to the fourth aspect wherein the plurality of grooves are formed all over a surface of the electrode.

According to a sixth aspect of the disclosure, there is provided the cell culture device according to any one of the first aspect to the fifth aspect wherein the plurality of grooves communicate with each other.

According to a seventh aspect of the disclosure, there is provided the cell culture device according to any one of the first aspect to the sixth aspect wherein the plurality of grooves are arranged on the surface of the electrode in a mesh manner.

According to an eighth aspect of the disclosure, there is provided the cell culture device according to any one of the first aspect to the seventh aspect wherein a groove width of the plurality of grooves is greater than 10 nm and less than 10 μm.

According to a ninth aspect of the disclosure, there is provided the cell culture device according to any one of the first aspect to the eighth aspect wherein a groove width of the plurality of grooves is equal to or less than 1/10 of a width of the contact hole.

According to a tenth aspect of the disclosure, there is provided a method for producing a cell culture device, the method including: disposing a wire on a main surface of a substrate; disposing, on the main surface of the substrate, an insulating film having a part that overlaps the wire; forming a contact hole in the insulating film at a position overlapping the wire; disposing, on the insulating film, an electrode having a part that overlaps the wire and the contact hole; and forming a plurality of grooves on at least a surface of a part of the electrode that overlaps the contact hole.

According to an eleventh aspect of the disclosure, there is provided the method for producing a cell culture device according to the tenth aspect wherein the plurality of grooves are formed by etching a surface of the electrode.

According to a twelfth aspect of the disclosure, there is provided the method for producing a cell culture device according to the eleventh aspect wherein the plurality of grooves are formed by etching the surface of the electrode by supplying an etchant containing iron(III) chloride to the electrode.

According to a thirteenth aspect of the disclosure, there is provided the method for producing a cell culture device according to the eleventh aspect wherein the plurality of grooves are formed by etching the surface of the electrode by supplying an etchant containing oxalic acid to the electrode.

### BRIEF DESCRIPTION OF THE DRAWINGS

FIG. 1 is a plan view of a cell culture device according to Embodiment 1;

FIG. 2 is a cross-sectional view of the cell culture device according to Embodiment 1 taken along line ii-ii in FIG. 1;

FIG. 3 is a plan view of a connection site between a wire and an electrode according to Embodiment 1;

FIG. 4 is a cross-sectional view of the cell culture device according to Embodiment 1 taken along line iv-iv in FIG. 3;

FIG. 5 is a photograph of the surface of the electrode according to Embodiment 1 magnified and taken by using an electron microscope or other devices;

FIG. 6 is the photograph of FIG. 5 in which a cell and axons are additionally drawn in the electrode according to Embodiment 1;

FIG. 7 is an enlarged cross-sectional view of a part of the electrode according to Embodiment 1 that overlaps a contact hole;

FIG. 8 is a cross-sectional view taken along line ii-ii in FIG. 1 in which wires are disposed in a method for producing the cell culture device according to Embodiment 1;

FIG. 9 is a cross-sectional view taken along line ii-ii in FIG. 1 in which contact holes are formed in an insulating film in the method for producing the cell culture device according to Embodiment 1;

FIG. 10 is a cross-sectional view taken along line ii-ii in FIG. 1 in which electrodes are formed in the method for producing the cell culture device according to Embodiment 1;

FIG. 11 is a cross-sectional view taken along line ii-ii in FIG. 1 in which the electrodes are surface-treated to form grooves in the method for producing the cell culture device according to Embodiment 1;

FIG. 12 is a cross-sectional view taken along line ii-ii in FIG. 1 in which a well is formed in the method for producing the cell culture device according to Embodiment 1;

FIG. 13 is a cross-sectional view of a cell culture device according to Embodiment 2 taken at the same cutting position as in FIG. 4;

FIG. 14 is a photograph of the surface of the electrode according to Embodiment 2 magnified and taken by using an electron microscope or other devices;

FIG. 15 is an enlarged cross-sectional view of a part of the electrode according to Embodiment 2 that overlaps a contact hole; and

FIG. 16 is a cross-sectional view taken at the same cutting position as in FIG. 2 in which the electrodes are surface-treated to form grooves in the method for producing the cell culture device according to Embodiment 2.

### DESCRIPTION OF THE EMBODIMENTS

Some embodiments of the technique disclosed herein will be described below. Matters (e.g., general matters related to cells to be cultured, techniques for culturing the cells, screening and preparation of pharmaceutical compositions, and general matters related to microfabrication techniques for producing cell culture devices) used to implement this technique, other than the matters specifically mentioned in this specification (e.g., the structure of the cell culture device disclosed herein), may be understood as matters of design by those skilled in the art based on the techniques known in the fields of cytology, physiology, medicine, pharmacology, biochemistry, genetic engineering, protein engineering, materials engineering, semiconductor engineering, ultra-precision machining, MEMS engineering, and other fields. This technique can be implemented based on the contents disclosed herein and the common technical knowledge in the art.

### Embodiment 1

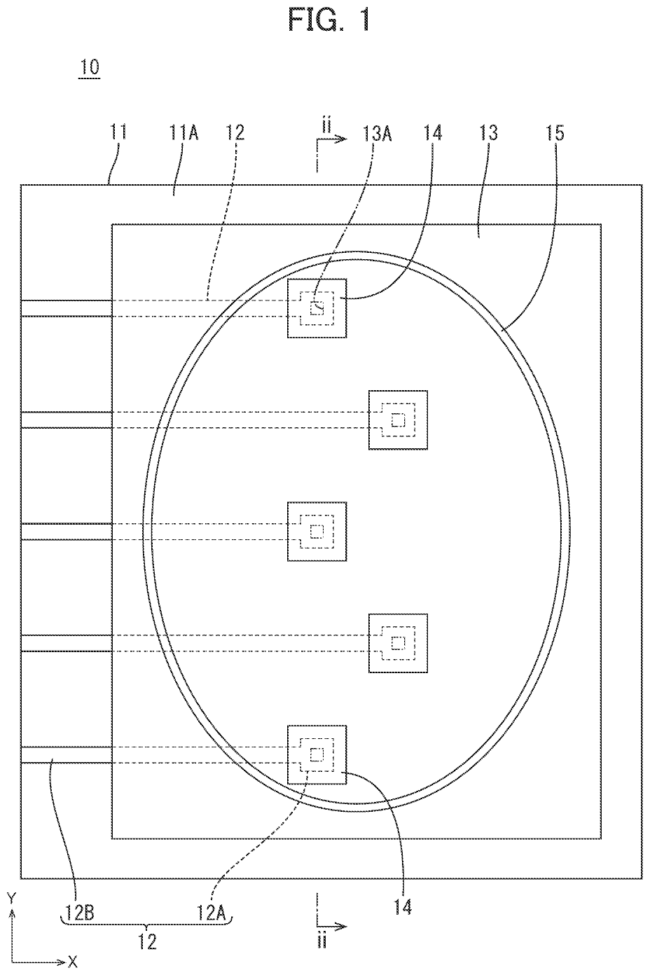

A cell culture device 10 according to Embodiment 1 will be described with reference to FIG. 1 to FIG. 11. The X-axis, Y-axis, and Z-axis are shown in some of the figures, and drawing is made such that each axis direction indicates the corresponding direction shown in the figures. The Z-axis direction in each figure corresponds to the thickness direction of the cell culture device 10.

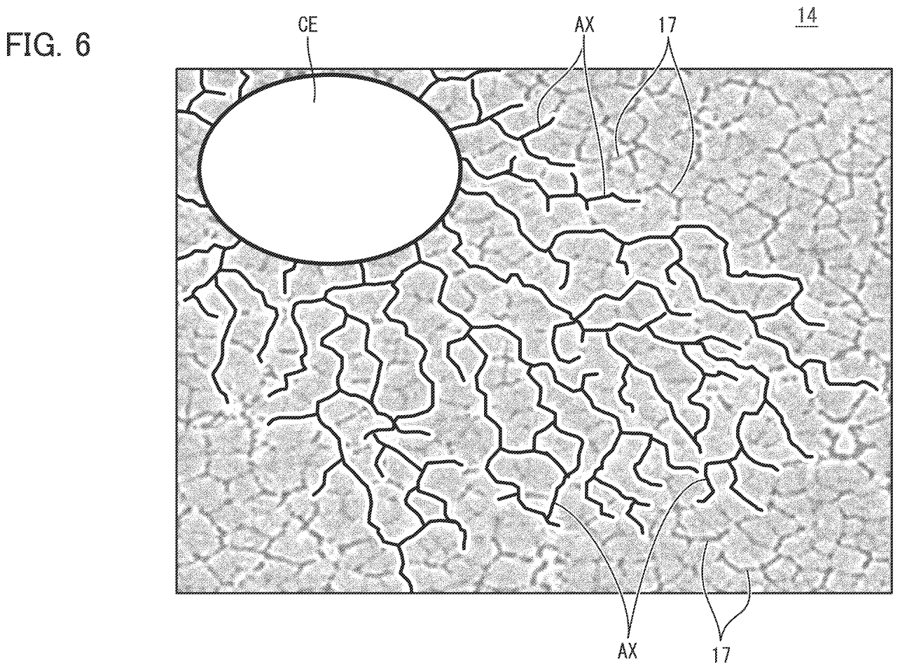

In the cell culture device 10 according to this embodiment, electrical signals (action potentials) generated in the electrical activity of the cell CE are recorded non-invasively and outside the cell CE while a cell CE (see FIG. 6) is cultured. This embodiment illustrates the cell culture device 10 mainly used to culture cells CE having axons AX like nerve cells.

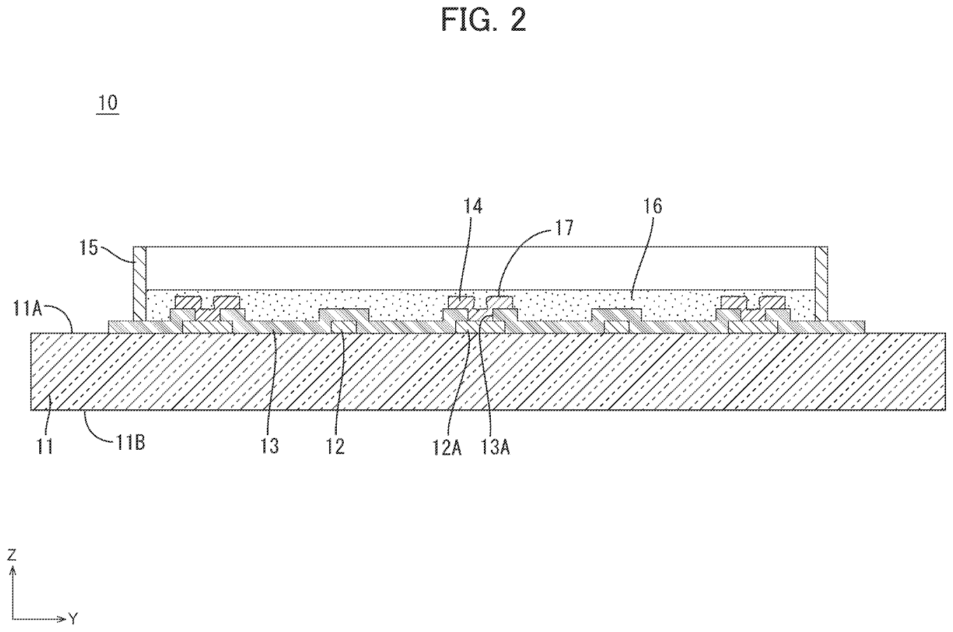

Referring to FIG. 1 and FIG. 2, the cell culture device 10 includes a substrate 11, wires 12, an insulating film 13, electrodes 14, and a well 15. The substrate 11 is an element that supports the wires 12, the insulating film 13, the electrodes 14, and other members. The substrate 11 may be a stage for supporting cells CE to be measured or for seeding and culturing cells CE. The substrate 11 of this embodiment has a flat plate shape. An upper main surface of a pair of main surfaces of the substrate 11 is a first main surface 11A constituting a culture plane on which the cells CE are cultured. A lower main surface of the pair of main surfaces of the substrate 11 is a second main surface 11B opposite the culture plane. The substrate 11 has a rectangular shape in plan view. A specific planar shape of the substrate 11 can be appropriately changed from the rectangular shape shown in FIG. 1. The substrate 11 is made of an insulating material. Examples of the insulating material include materials having a volume resistivity of 10⁷ Ωcm or more (e.g., 10¹⁰ Ωcm or more, 10¹² Ωcm or more, or 10¹⁵ Ωcm or more) at room temperature (e.g., 25° C.). The insulating material may be, for example, an organic or inorganic material having the above volume resistivity. The substrate 11 may be made of, but not limited to, a light-transmissive material, e.g., a transparent material, so that the cells CE on the first main surface 11A side can be observed from the second main surface 11B side across the substrate 11. The substrate 11 may be colorless and transparent.

Examples of the material of the substrate 11 include various glasses and synthetic resins. Examples of glasses include soda-lime glass, borosilicate glass, and quartz glass. The glass may be, but not limited to, an alkali-free glass that contains 0.1 mass % or less of alkali components in terms of oxides and in which the elution of alkali ions is highly suppressed. Examples of synthetic resins include polydimethylsiloxane (PDMS), polystyrene, polypropylene, polyethylene terephthalate (PET), polymethyl methacrylate, nylon, and polyurethane, which have a relatively high volume resistivity (e.g., 10¹⁰ Ωcm or more, 10¹² Ωcm or more, or 10¹⁵ Ωcm or more) and have biocompatibility. The substrate 11 may have any thickness, for example, about 0.2 mm to 1 mm (e.g., 0.5 mm, 0.7 mm).

Referring to FIG. 1 and FIG. 2, the wires 12 are disposed on the first main surface 11A of the substrate 11. The wires 12 are formed in a predetermined wiring pattern by pattering a conductive film on the first main surface 11A by known photolithography. The thickness of the wires 12 composed of the conductive film is, for example, about in the range of 50 nm to 1000 nm. The wires 12 extend in the X-axis direction and span from one edge (on the left side in FIG. 1) to near the center of the substrate 11 in the X-axis direction. The wires 12 are covered by the insulating film 13 near the center of the substrate 11 in the X-axis direction and exposed near one edge of the substrate 11 in the X-axis direction without being covered by the insulating film 13. The opposite ends of each wire 12 in the X-axis direction includes a first end portion 12A located in the range in which the insulating film 13 is formed (near the center of the substrate 11 in the X-axis direction), and a second end portion 12B located out of the range in which the insulating film 13 is formed. The wires 12 other than the first end portions 12A have a substantially uniform wire width (dimension in the Y-axis direction). The first end portions 12A are wider, or larger in dimension in the Y-axis direction, than the other portions (including the second end portions 12B) of the wires 12. The first end portions 12A have a rectangular shape in plan view. The wires 12 are arranged parallel to each other. In FIG. 1, the number of the wires 12 is 5. The number of the wires 12 can be appropriately changed from that illustrated in FIG. 1. There are two types of wires 12 different in length in the X-axis direction. The relatively longer wires 12 alternate with the relatively shorter wires 12 in the Y-axis direction. In other words, two wires 12 adjacent to each other in the Y-axis direction are different in length in the X-axis direction. Accordingly, the first end portions 12A of the wires 12 are staggered from each other in plan view.

The conductive film constituting the wires 12 is made of a conducting material (conductive material) having electrical conductivity. Examples of the conducting material include metal materials, conductive resin materials, and conductive inorganic materials. From the viewpoint of high thermal stability and high electrical conductivity, metal materials may be used. The metal material used for the conductive film may be any one metal selected from gold (Au), silver (Ag), copper (Cu), titanium (Ti), aluminum (Al), nickel (Ni), chromium (Cr), molybdenum (Mo), niobium (Nb), tantalum (Ta), and tungsten (W), an alloy containing the metal, an alloy containing two or more of these metals, or other metals. Having high electrical conductivity, metals containing these elements allow the wires 12 to have low resistivity even when the wires 12 are minimized. Examples of the metal material used for the conductive film include Au, Ag, Cu, W, Ti, Al, TaN (tantalum nitride), and MoW (molybdenum tungsten alloy). Since the wires 12 are disposed on the first main surface 11A of the substrate 11, a metal having a relatively high melting point, such as Ta, W, Mo, Ni, or Ti, may be added to the metal material of the conductive film. The conductive film may have a multilayer structure, such as W/TaN, Ti/Al/Ti, or Cu/Ti in order from the upper layer side, to obtain both low resistance and close contact with a base layer (e.g., substrate). Examples of the metal material constituting the conductive film located in regions that may be in contact with the cells CE include Au and Ti, which have low cytotoxicity. The conductive film may have, for example, a monolayer structure composed of a MoW alloy with low resistance to reduce the wire resistance. The wires 12 in this embodiment are composed of the conductive film containing any of these metal materials. The metal material contained in the conductive film constituting the wires 12 has light-shielding properties. Therefore, the wires 12 have light-shielding properties.

Referring to FIG. 1 and FIG. 2, the insulating film 13 is disposed on the first main surface 11A of the substrate 11, and a part of the insulating film 13 overlaps the wires 12 from above. The insulating film 13 selectively covers part of the substrate 11. Specifically, the insulating film 13 covers a central part of the substrate 11 but does not cover a peripheral part of the substrate 11. Portions of the wires 12 (including the first end portions 12A) located in a central part of the substrate 11 are covered by the insulating film 13, so that short-circuiting between adjacent wires 12 is unlikely to occur. Portions of the wires 12 (including the second end portions 12B) located in a peripheral part of the substrate 11 are exposed without being covered by the insulating film 13. The second end portions 12B, which are exposed portions of the wires 12, can be brought into contact with, for example, a probe pin of a measuring device (e.g., prober). With this configuration, the potential of each wire 12 can be output to the measuring device. The thickness of the insulating film 13 is, for example, about in the range of 300 nm to 1000 nm.

The insulating film 13 can be made of the same electrically insulating material as that of the substrate 11 and may be made of any electrically insulating material stable in a cell culture environment. The insulating film 13 may be made of, but not limited to, an inorganic material, such as silicon nitride (e.g., Si₃N₄), silicon oxide (e.g., SiO₂), or silicon oxynitride (e.g., Si₂N₂O) to insulate the wires 12. The representative compositions of the materials are shown in the parentheses, but the compositions of the materials are not limited to these. In addition, the insulating film 13 may be made of an organic material, such as an acrylic resin or a polyimide resin. The insulating film 13 may have a monolayer structure composed of any one of these materials, or may have a multilayer structure composed of two or more of these materials. The insulating film 13 may be made of, but not limited to, a light-transmissive material, e.g., a transparent material, so as to observe the cells CE. The insulating film 13 may be colorless and transparent.

Referring to FIG. 1 and FIG. 2, the electrodes 14 are disposed on the insulating film 13 and overlap the wires 12. The electrodes 14 are formed in a predetermined electrode pattern by pattering the conductive film on the insulating film 13 by known photolithography. The thickness of the electrodes 14 composed of the conductive film is, for example, about in the range of 50 nm to 150 nm. A part of each electrode 14 overlaps the first end portion 12A of the corresponding wire 12. The electrodes 14 each have a rectangular shape in plan view and have larger dimensions (outside dimensions in plan view) in the X-axis direction and the Y-axis direction than the corresponding first end portion 12A. In other words, the electrodes 14 have a larger area than the first end portions 12A. The electrodes 14 are concentric with the first end portions 12A in plan view. A central part of each electrode 14 is an overlap portion 14A that overlaps the corresponding first end portion 12A. A peripheral part of each electrode 14 is a non-overlap portion 14B that does not overlap the corresponding first end portion 12A. The electrodes 14 are spaced apart from each other in the range in which the insulating film 13 is formed. The electrodes 14 are staggered from each other in plan view, and the arrangement of the electrodes 14 corresponds to the arrangement of the first end portions 12A.

The conductive film constituting the electrodes 14 is made of a conducting material having electrical conductivity. Examples of the conducting material include conductive inorganic materials. Examples of conductive inorganic materials include semiconductor oxides (may be metal oxides) having a bandgap of 3 eV or more, such as tin oxide (SnO₂; including tin oxide doped with Sb (antimony), Ta, F (fluorine), or other elements), zinc oxide (ZnO; including zinc oxide doped with Al, Ga (gallium), or other elements), indium tin oxide (ITO), indium zinc oxide (IZO), indium gallium zinc oxide (IGZO). These semiconductor oxides are confirmed to be transparent and biologically inert. In particular, the conductive film constituting the electrodes 14 may be made of a material transparent to visible light, like the substrate 11 and the insulating film 13, because the use of such a material avoids the electrodes 14 from hindering observation of the cultured cells CE from the second main surface 11B side of the substrate 11 across the substrate 11. The electrodes 14 in this embodiment are composed of, for example, ITO. The use of such a material can form a stable conductive film with low cytotoxicity. The conducting material of the conductive film constituting the electrodes 14 may be, for example, a conductive resin material. Examples of the conductive resin material include conductive polyacetylene, conductive polythiophene, conductive polyaniline, and conductive polyethylenedioxythiophene (PEDOT).

Referring to FIG. 1 and FIG. 2, the well 15 is disposed on the insulating film 13. The well 15 is a wall erecting from the surface of the insulating film 13 and has a substantially annular shape in plan view. The inner space of the well 15 having a substantially annular shape is a region in which the cells CE can be cultured, that is, a cell culture region. It can be said that the well 15 defines the cell culture region. The well 15 can hold a broth and other materials in the cell culture region without leakage to the outside. The well 15 is located on a central part of the insulating film 13 to enclose all of the electrodes 14. In other words, all of the electrodes 14 are located in the cell culture region. The well 15 may have any height unless the broth in the cell culture region leaks to the outside.

The well 15 can be made of the same electrically insulating material as that of the substrate 11 and may be made of any electrically insulating material stable in a cell culture environment. The well 15 is made of, but not limited to, a photocurable resin material, a silicone, a glass material, or other materials. When the well 15 is made of a silicone, for example, dimethylpolysiloxane (PDMS) can be used. The well 15 may be composed of an adhesive or tape attached to the surface of the insulating film 13. Any material with low cytotoxicity selected from the above materials may be used for the well 15. The well 15 may be made of, but not limited to, a light-transmissive material, e.g., a transparent material, so as to observe the cells CE. The well 15 may be colorless and transparent.

In the cell culture device 10 having the above structure, a coating material 16 is applied to the inside of the cell culture region after the inside of the cell culture region is sterilized before actually culturing the cells CE. The coating material 16 will be described. Referring to FIG. 2, the coating material 16 is disposed on the insulating film 13 and the electrodes 14. The coating material 16 is located in the cell culture region enclosed by the well 15 and helps the cells CE to attach to the surfaces of the insulating film 13 and the electrodes 14. The coating material 16 is disposed by surface-treating the insulating film 13 and the electrodes 14 in the cell culture region. The coating material 16 constitutes the culture medium for culturing the cells CE in vitro. The coating material 16 is composed of an extracellular matrix (ECM). The extracellular matrix is an extracellular matrix suitable for the cells CE and has high affinity for the cells CE to improve the attachment of the cells CE. Examples of the extracellular matrix include proteins, such as collagen, laminin, fibronectin, vitronectin, and gelatin. The coating material 16 in the cell culture region can promote the growth of the cultured cells CE.

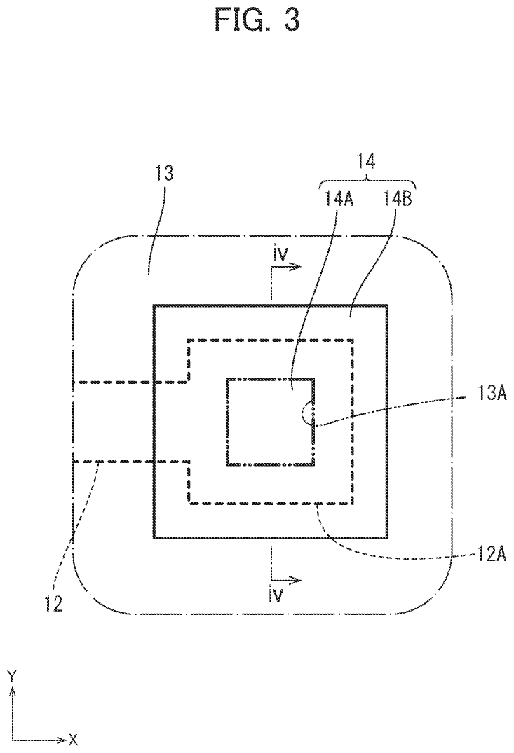

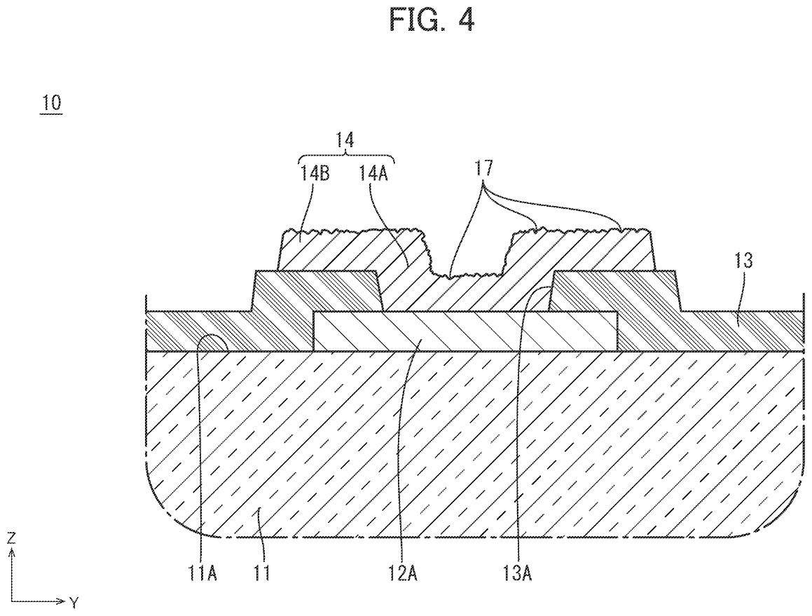

The configuration of the wire 12, the insulating film 13, and the electrode 14 will be described in detail. Referring to FIG. 3 and FIG. 4, the insulating film 13 has a contact hole 13A at a position overlapping both the wire 12 and the electrode 14. The electrode 14 is electrically connected to the wire 12 on the lower side through the contact hole 13A. With this configuration, the electrical signals from the cells CE cultured in the cell culture region are transmitted from the electrode 14 to the wire 12. The electrical signals transmitted to the wire 12 are measured with a measuring device through, for example, a probe pin in contact with the portion (second end portion 12B) of the wire 12 exposed from the insulating film 13. The contact hole 13A is concentric with both the first end portion 12A of the wire 12 and the electrode 14 in plan view. The contact hole 13A has smaller dimensions in the X-axis direction and the Y-axis direction than the first end portion 12A. A part (central part) of the overlap portion 14A of the electrode 14 is in direct contact with a part (central part) of the first end portion 12A of the wire 12 through the contact hole 13A. It can be said that the first end portion 12A is a contact portion that establishes connection between the wire 12 and the electrode 14. The remaining part (peripheral part) of the overlap portion 14A of the electrode 14, together with the non-overlap portion 14B, is stacked on the upper side of the insulating film 13. The non-overlap portion 14B of the electrode 14 protrudes from the first end portion 12A of the wire 12 to the outside in plan view. The dimensions of the contact hole 13A (widths of the contact hole 13A) in the X-axis direction and the Y-axis direction are, for example, about in the range of 10 μm to 100 μm. The contact hole 13A is formed in a predetermined pattern by pattering the insulating film 13 by known photolithography.

Referring to FIG. 4, grooves 17 are formed on at least the surface of a part (a central part of the overlap portion 14A) of the electrode 14 having the above configuration, the part overlapping the contact hole 13A. The grooves 17 are also formed on the surface of a part of the electrode 14 that does not overlap the contact hole 13A (the entire region of the peripheral part of the overlap portion 14A and the non-overlap portion 14B), in addition to a part of the electrode 14 that overlaps the contact hole 13A. In other words, the grooves 17 are formed on the entire surface of the electrode 14.

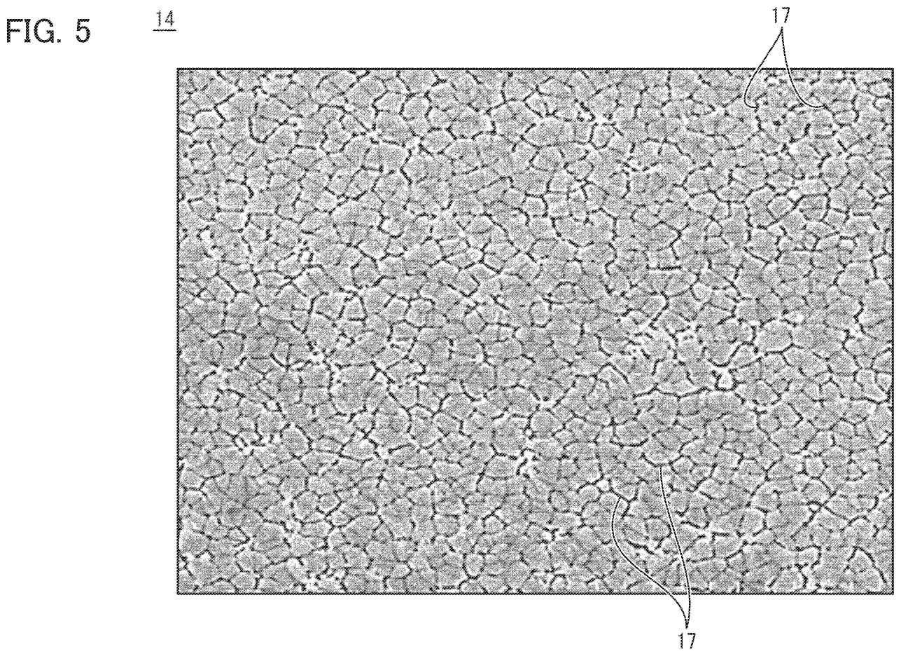

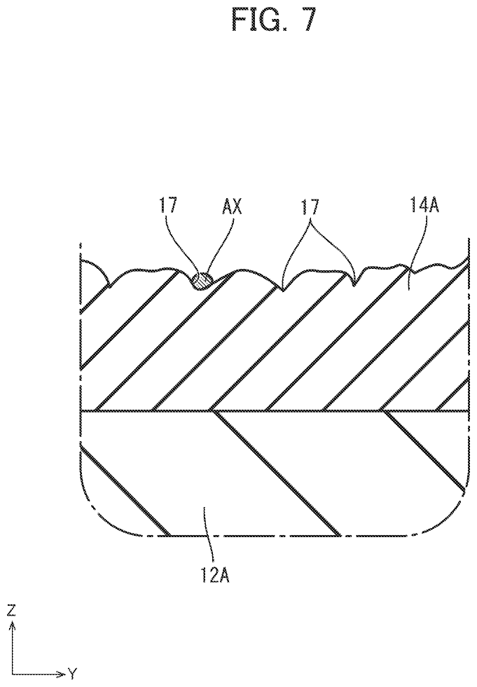

The configuration of the grooves 17 will be described in detail with reference to FIG. 5 to FIG. 7. FIG. 5 is a photograph of the surface of an electrode 14 magnified and taken by using an electron microscope or other devices. FIG. 6 is the photograph of FIG. 5 in which a cell CE and axons AX are additionally drawn. FIG. 7 is an enlarged cross-sectional view of a part of the electrode 14 that overlaps a contact hole 13A. Reference to FIG. 5 to FIG. 7, a countless number of the grooves 17 are arranged on the surface of the electrode 14 in a mesh manner. The grooves 17 arranged on the surface of the electrode 14 communicate with each other. The paths of the grooves 17 are randomly arranged in plan view. The grooves 17 each have a depth smaller than the thickness of the electrode 14. Thus, the wire 12 on the lower side of the electrode 14 is not exposed through the grooves 17 in this embodiment. In other words, the grooves 17 are not deep enough to reach the wire 12 on the lower side of the electrode 14. The grooves 17 do not necessarily have a uniform depth and may vary in depth according to their positions on the surface of the electrode 14. The grooves 17 have a groove width of, for example, about several tens of nanometers.

Since the grooves 17 are formed on at least the surface of a part of the electrode 14 that overlaps the contact hole 13A, the surface area is larger than that in a case where the grooves 17 are not formed. This configuration increases the contact area between the cultured cell CE and the electrode 14 and thus sufficiently increases the signal intensity of the electrical signals measured with a measuring device, resulting in high measurement sensitivity. In addition, the grooves 17 are present on at least a part of the electrode 14 that overlaps the contact hole 13A, that is, a part of the electrode 14 that is in direct contact with the wire 12 on the lower side. When the grooves 17 are formed on the surface of the electrode 14, the grooves 17 may have various depths, and some grooves 17 may be deep to reach the wire 12. In this case, the electrode 14 may be divided by the grooves 17 to generate island portions. Even if the island portions are generated, the island portions are in direct contact with the wire 12 through the contact hole 13A, and the electrical signals from the cell CE in contact with the island portions can be effectively transmitted to the wire 12. With this configuration, the measurement sensitivity is unlikely to decrease because of generation of the island portions.

Furthermore, the electrode 14 has the overlap portion 14A, which is a part of the electrode 14, overlapping the wire 12 and has the non-overlap portion 14B, which is the remaining part, not overlapping the wire 12. As long as the cultured cell CE is present on the surface of the non-overlap portion 14B of the electrode 14, the wire 12 is less likely to hinder observation of the cell CE with an electron microscope or other devices. Since the substrate 11, the insulating film 13, and the electrode 14 are all light-transmissive, it is easy to observe the cell CE cultured on the surface of the electrode 14 across the substrate 11 even when the wire 12 has light-shielding properties.

Since the grooves 17 each have a depth smaller than the thickness of the electrode 14, the axons AX of the cell CE cultured on the surface of the electrode 14 can be grown along the grooves 17. Therefore, the cell CE having the axons AX like a nerve cell can be grown. Since the grooves 17 are formed on the entire surface of the electrode 14, the growth of the cell CE cultured on the surface of the electrode 14 can be promoted over the entire surface of the electrode 14. This cell culture device is easier to produce than a cell culture device in which the grooves 17 are formed on only parts of the electrodes 14.

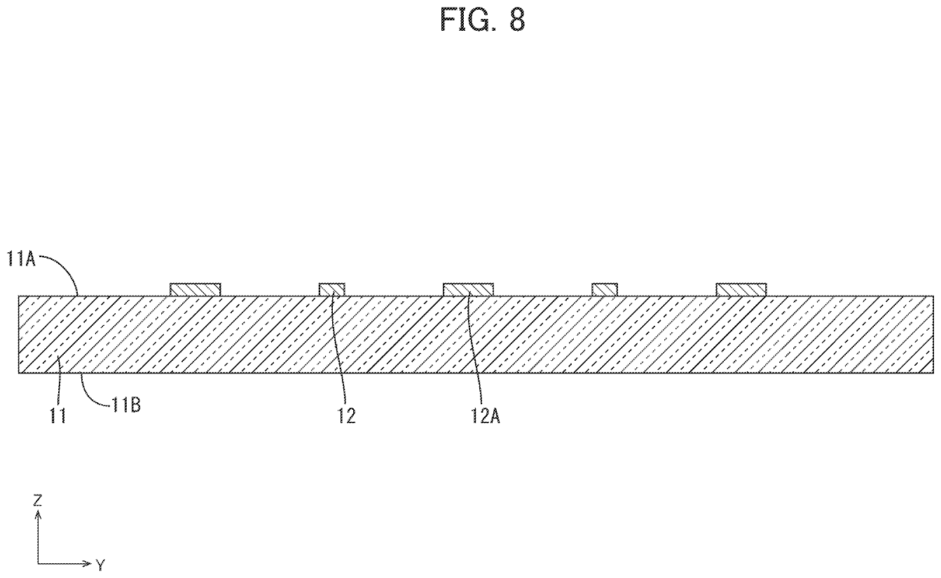

The cell culture device 10 according to this embodiment has the above structure. Next, a method for producing the cell culture device 10 will be described. To produce the cell culture device 10, first, a conductive film constituting the wires 12 is formed on the first main surface 11A of the substrate 11. When the wires 12 have a multilayer structure composed of multiple conductive films (e.g., Ti/Al/Ti), the conductive films are formed continuously. Subsequently, a photoresist film is applied to the conductive film, and the photoresist film is then exposed and developed by using an exposure device and a photomask. The conductive film is etched using the developed photoresist film so that the conductive film is patterned to form multiple wires 12 as illustrated in FIG. 8. Subsequently, the photoresist film is removed by ashing.

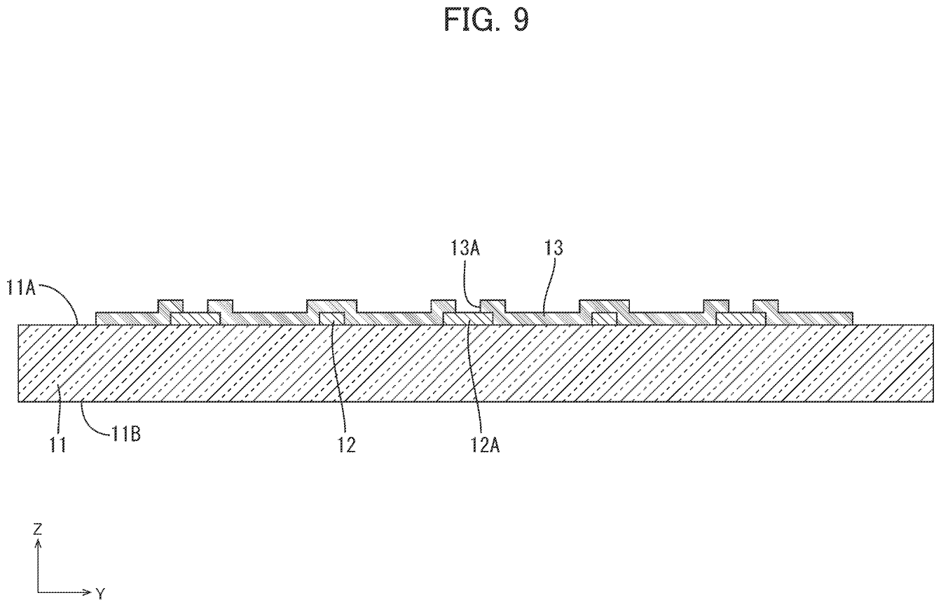

After the wires 12 are formed, the insulating film 13 is formed on the first main surface 11A of the substrate 11. Subsequently, a photoresist film is applied to the insulating film 13, and the photoresist film is then exposed and developed by using an exposure device and a photomask. The insulating film 13 is etched using the developed photoresist film to pattern the insulating film 13 as illustrated in FIG. 9. The insulating film 13 selectively remains only in a central part of the substrate 11, and the contact holes 13A are formed at positions overlapping the respective first end portions 12A of the wires 12. Subsequently, the photoresist film is removed by ashing.

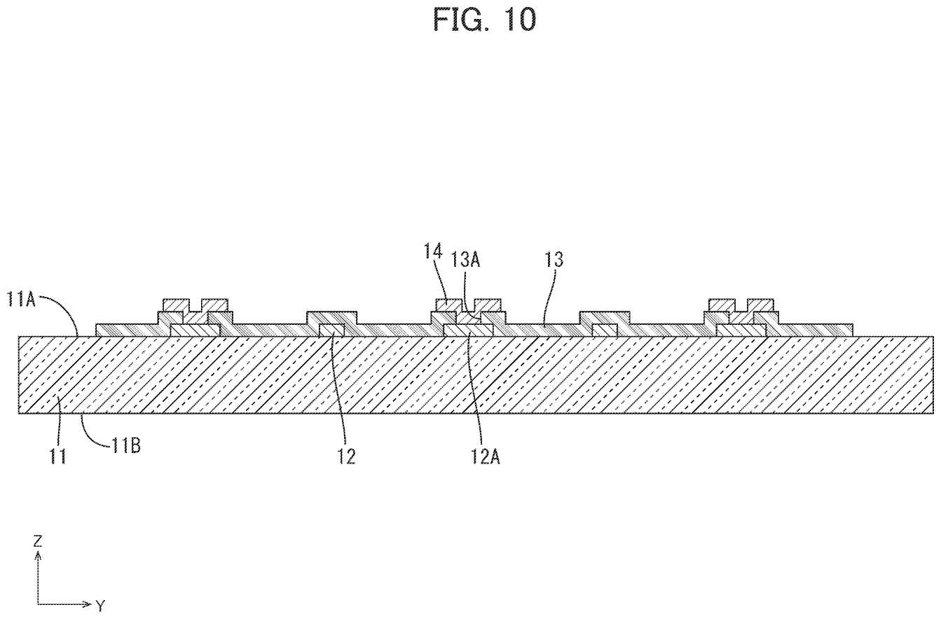

After the insulating film 13 is patterned, a conductive film constituting the electrodes 14 is formed on the insulating film 13. In this embodiment, ITO is used as a material of the conductive film. The conductive film constituting the electrodes 14 is formed by, for example, sputtering. A photoresist film is applied to the conductive film, and the photoresist film is then exposed and developed by using an exposure device and a photomask. The conductive film is etched using the developed photoresist film to pattern the insulating film as illustrated in FIG. 10. This forms multiple electrodes 14. The electrodes 14 are annealed (heated, crystalized) to promote crystallization of ITO, which is a material of the conductive film. The annealing treatment is performed, for example, in an environment with a temperature of about 150° C. to 250° C. for about 30 to 60 minutes.

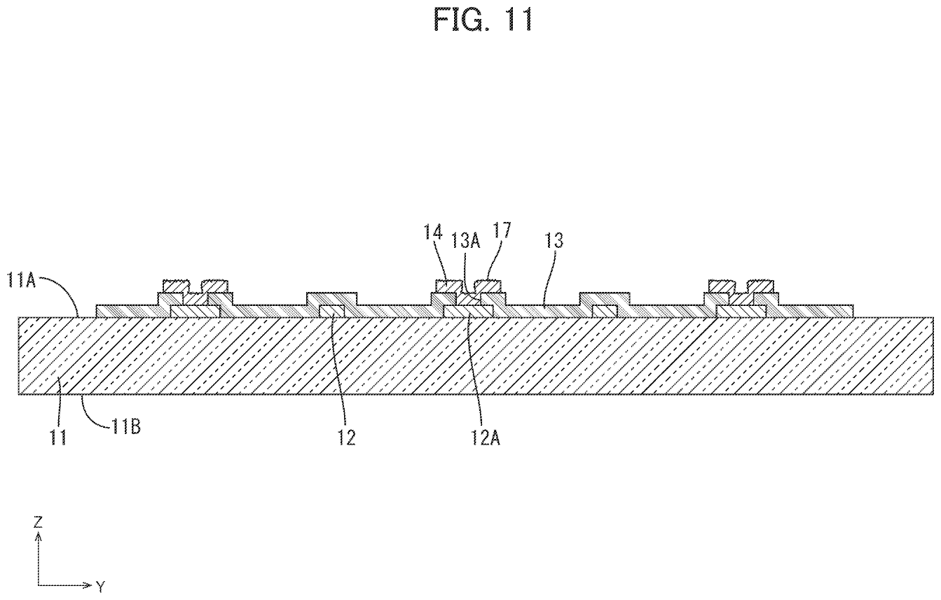

The electrodes 14 on the insulating film 13 are surface-treated (etched) by using an etchant containing iron(III) chloride. In the surface treatment, the electrodes 14 are dipped in an aqueous solution of iron(III) chloride, which is an etchant. The dipping time of the electrodes 14 in an aqueous solution of iron(III) chloride is, for example, about 60 seconds. The surface-treated electrodes 14 are washed with pure water or other liquids. Referring to FIG. 11, multiple grooves 17 are formed on the surface of the surface-treated electrodes 14. The formed grooves 17 are arranged on the entire surface of the electrodes 14 in a mesh manner, and the grooves 17 more assuredly have a depth smaller than the thickness of the electrodes 14 (see FIG. 5 to FIG. 7).

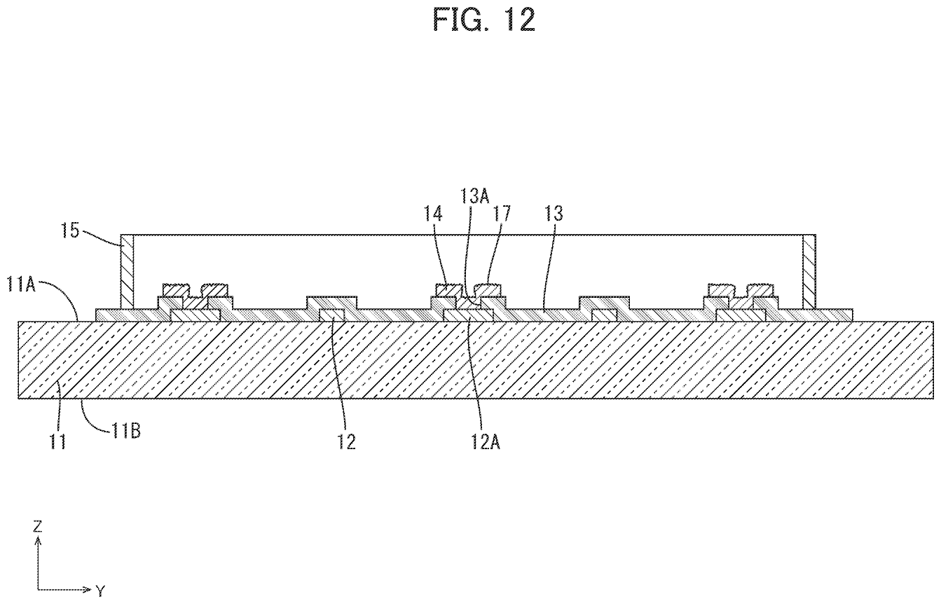

Referring to FIG. 12, the well 15 is formed on the insulating film 13 after the electrodes 14 are formed. A specific process for forming the well 15 depends on the material (e.g., photocurable resin material, silicone, glass material, adhesive, tape) of the well 15. The cell culture device 10 is produced accordingly. Before the cells CE are cultured using the produced cell culture device 10, the inside of the cell culture region defined by the well 15 is sterilized. Subsequently, the coating material 16 is applied to the inside of the cell culture region (see FIG. 2). After the excess coating material is removed, the inside of the cell culture region is filled with a culture medium, and the cells CE are seeded in the cell culture region. While the cells CE are cultured, the electrical signals from the cells CE are measured by using a measuring device. The cell culture device 10 of this embodiment enables culturing of the cells CE in parallel with measurement of electrical signals from the cells CE and thus reduces the need for transfer of the cultured cells CE compared with the case of using a device for temporarily culturing of the cells CE and a device for measuring electrical signals from the cells CE. Thus, there is no need of the work for transferring the cells CE, and the damage to the cells CE caused by the transfer work can be avoided.

As described above, the cell culture device 10 of this embodiment includes the substrate 11, the wires 12 disposed on the first main surface (main surface) 11A of the substrate 11, the insulating film 13 disposed on the first main surface 11A of the substrate 11 and having a part that overlaps the wires 12, and the electrodes 14 disposed on the insulating film 13 and having a part that overlaps the wires 12, wherein the insulating film 13 has the contact holes 13A at positions overlapping the wires 12 and the electrodes 14, and the grooves 17 are formed on at least the surface of a part of each electrode 14 that overlaps the corresponding contact hole 13A.

Since the grooves 17 are formed on at least the surface of a part of each electrode 14 that overlaps the corresponding contact hole 13A, the surface area is larger than that in a case where the grooves 17 are not formed. This configuration increases the contact area between the cultured cells CE and the electrodes 14 and results in high measurement sensitivity. In addition, the grooves 17 are present on at least a part of each electrode 14 that overlaps the corresponding contact hole 13A. If the grooves 17 are deep enough to reach the wires 12 so that part of each electrode 14 forms islands, the measurement through the island portions become effective upon contact of the island portions with the wires 12 according to this configuration. This avoids reducing measurement sensitivity. Furthermore, part of each electrode 14 overlaps the corresponding wire 12, and the remaining part does not overlap the corresponding wire 12. As long as the cultured cells CE are present on the surface of a part of each electrode 14 that does not overlap the corresponding wire 12, the wires 12 are less likely to hinder observation of the cells CE with an electron microscope or other devices. For example, when multiple wires 12 are disposed on the first main surface 11A of the substrate 11, the insulating film 13 can avoid short-circuiting between the wires 12.

The substrate 11 and the insulating film 13 are each made of a light-transmissive insulating material, the wires 12 are made of a light-shielding conducting material, and the electrodes 14 are made of a light-transmissive conducting material. Since the substrate 11, the insulating film 13, and the electrodes 14 are all light-transmissive, it is easy to observe the cells CE cultured on the surface of the electrodes 14 across the substrate 11 even when the wires 12 have light-shielding properties.

The grooves 17 each have a depth smaller than the thickness of the electrodes 14. The axons AX of the cells CE cultured on the surfaces of the electrodes 14 can be grown along the grooves 17.

The grooves 17 are formed on the entire surface of each electrode 14. With the grooves 17, the growth of the cells CE cultured on the surface of the electrodes 14 can be promoted over the entire surface of the electrodes 14. This cell culture device is easier to produce than a cell culture device in which the grooves 17 are formed on only parts of the electrodes 14.

A method for producing the cell culture device 10 of this embodiment includes: disposing the wires 12 on the first main surface 11A of the substrate 11; disposing, on the first main surface 11A of the substrate 11, the insulating film 13 having a part that overlaps the wires 12; forming the contact holes 13A in the insulating film 13 at positions overlapping the wires 12; disposing, on the insulating film 13, the electrodes 14 each having a part that overlaps the corresponding wire 12 and the corresponding contact hole 13A; and forming the grooves 17 on at least the surface of a part of each electrode 14 that overlaps the corresponding contact hole 13A.

Since the grooves 17 are formed on at least the surface of a part of each electrode 14 that overlaps the corresponding contact hole 13A in the cell culture device 10 thus produced, the surface area is larger than that in a case where the grooves 17 are not formed. This configuration increases the contact area between the cultured cells CE and the electrodes 14 and results in high measurement sensitivity. In addition, the grooves 17 are present on at least a part of each electrode 14 that overlaps the corresponding contact hole 13A. If the grooves 17 are deep enough to reach the wires 12 so that part of each electrode 14 forms islands, the measurement through the island portions become effective upon contact of the island portions with the wires 12 according to this configuration. This avoids reducing measurement sensitivity. Furthermore, part of each electrode 14 overlaps the corresponding wire 12, and the remaining part does not overlap the corresponding wire 12. As long as the cultured cells CE are present on the surface of a part of each electrode 14 that does not overlap the corresponding wire 12, the wires 12 are less likely to hinder observation of the cells CE with an electron microscope or other devices. For example, when multiple wires 12 are disposed on the first main surface 11A of the substrate 11, the insulating film 13 can avoid short-circuiting between the wires 12.

The grooves 17 are formed by etching the surface of the electrodes 14 by supplying an etchant containing iron(III) chloride to the electrodes 14. When the etchant containing iron(III) chloride is supplied to the electrodes 14, the surface of the electrodes 14 is etched to form the grooves 17. The grooves 17 thus formed each have a depth smaller than the thickness of the electrodes 14. Accordingly, the axons AX of the cells CE cultured on the surfaces of the electrodes 14 can be grown along the grooves 17.

### Embodiment 2

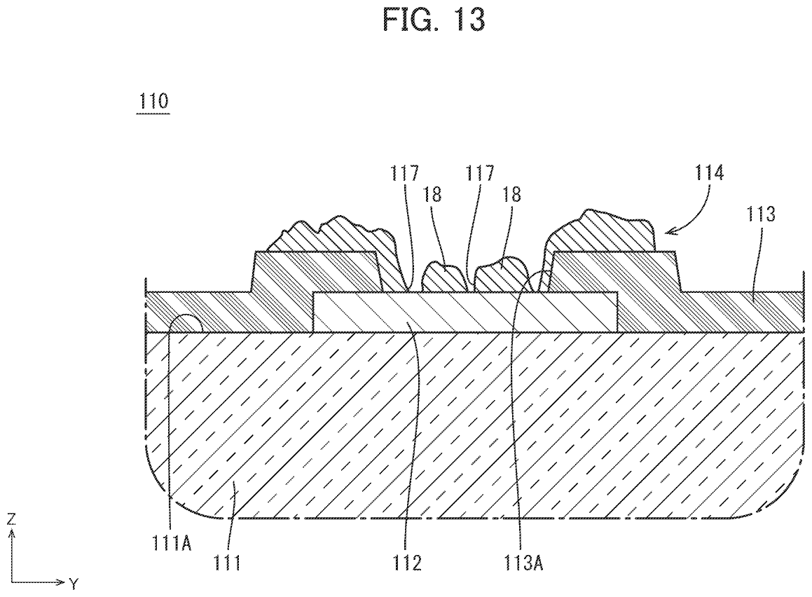

Embodiment 2 will be described with reference to FIG. 13 to FIG. 16. In Embodiment 2, the configuration of grooves 117 and other features are changed. The overlapping description on the structure, operation, and effect that are the same as those in Embodiment 1 will be omitted.

Referring to FIG. 13, the grooves 117 formed on the surface of an electrode 114 according to this embodiment have a depth equal to the thickness of the electrode 114. Thus, a wire 112 on the lower side of the electrode 114 is partially exposed through the grooves 117 in this embodiment. In other words, the grooves 117 are deep enough to reach the wire 112 on the lower side of the electrode 114. The electrode 114 is divided by these grooves 117 and includes multiple island portions 18. The island portions 18 are physically separated from each other by the grooves 117. The wire 112 is thus partially exposed through the grooves 117 when the electrode 114 includes the island portions 18.

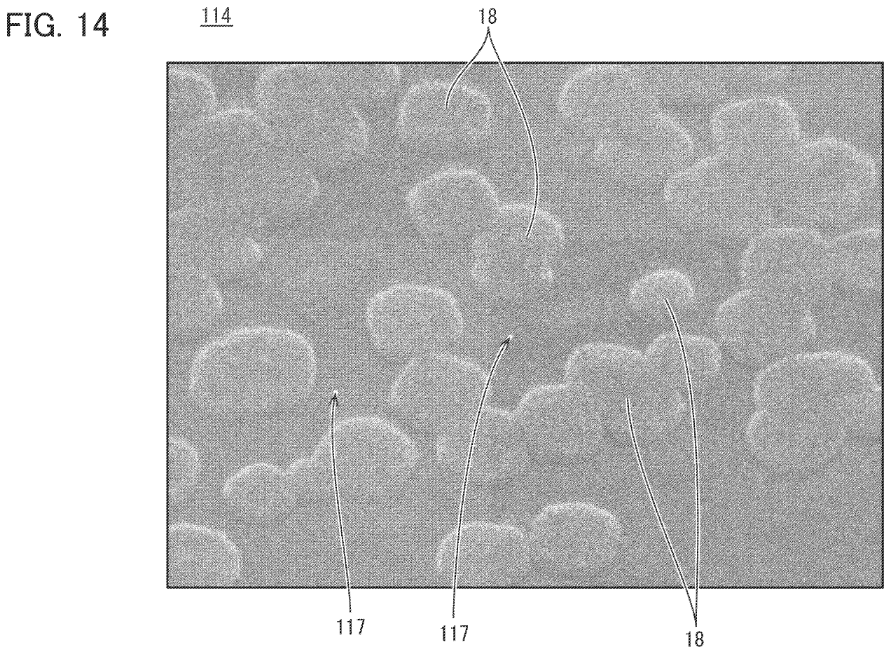

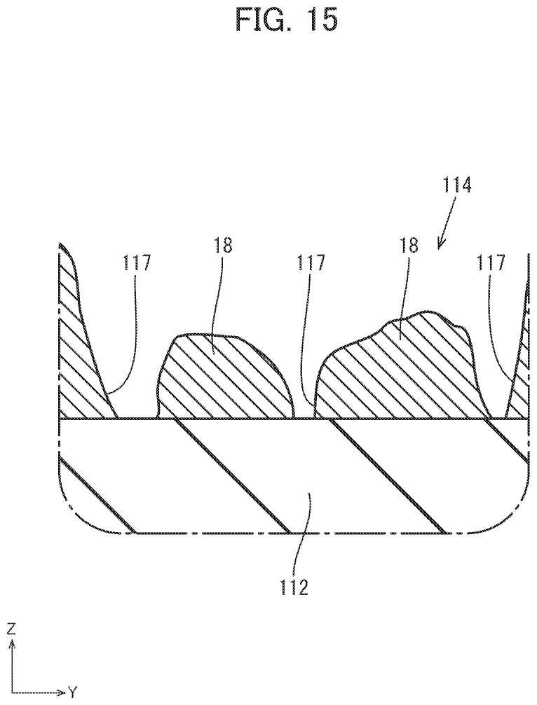

The configuration of the grooves 117 will be described in detail with reference to FIG. 14 and FIG. 15. FIG. 14 is a photograph of the surface of the electrode 114 magnified and taken by using an electron microscope or other devices. FIG. 15 is an enlarged cross-sectional view of a part of the electrode 114 that overlaps a contact hole 113A. Reference to FIG. 14 and FIG. 15, a countless number of the grooves 117 are arranged on the entire surface of the electrode 114 in a mesh manner, and the mesh of the grooves 117 is coarser than that of the grooves 17 described above in Embodiment 1 (see FIG. 5). The island portions 18 generated by the grooves 117 are random in size and shape in plan view and each generally have a planar shape with a curved surface. The groove width of the grooves 117, that is, the spacing between the adjacent island portions 18, is, for example, about in the range of several tens of nanometers to several micrometers. The groove width of the grooves 117 according to this embodiment is generally larger than the groove width of the grooves 17 described in Embodiment 1. With this configuration, the cells CE cultured on the surface of the electrode 114 can be grown and spread out in a plane along the grooves 117 separating the island portions 18. The island portions 18 include multiple island portions 18 in direct contact with the wire 112 through the contact hole 113A. As long as the island portions 18 in direct contact with the wire 112 is in contact with a cell CE, the electrical signals from the cell CE can be transmitted from the island portions 18 to the wire 112. In other words, the measurement through the island portions 18 become effective. With this configuration, the measurement sensitivity is unlikely to decrease even when the electrode 114 includes the island portions 18. The grooves 117 do not necessarily have a uniform depth and may vary in depth according to their positions on the surface of the electrode 114.

A cell culture device 110 according to this embodiment has the above structure. Next, a method for producing the cell culture device 110 will be described. The wires 112, an insulating film 113, and the electrodes 114 are sequentially disposed on a first main surface 111A of a substrate 111 by the same procedure as in Embodiment 1 (see FIG. 8 to FIG. 10). The electrodes 114 on the insulating film 113 in this embodiment are annealed, for example, in an environment with a temperature of about 220° C. for about 10 minutes. In other words, the treatment time in the annealing treatment of the electrodes 114 in this embodiment is much shorter than the treatment time (e.g., 40 minutes) in Embodiment 1. Specifically, the treatment time in this embodiment is, for example, about ¼ the treatment time in Embodiment 1. Therefore, the degree of crystallization of ITO contained in the electrodes 114 in this embodiment is lower than that in Embodiment 1.

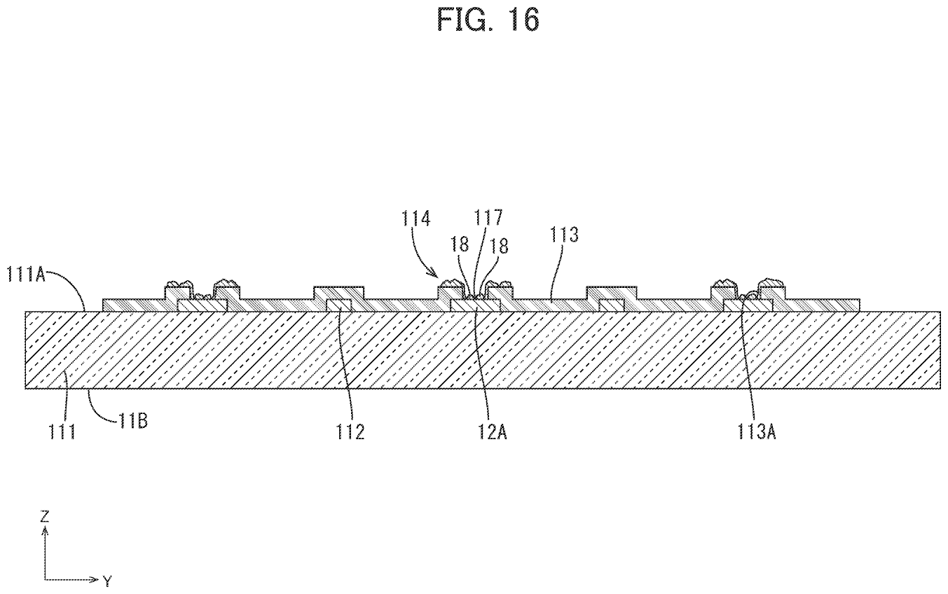

The electrodes 114 on the insulating film 113 are surface-treated (etched) by using an etchant containing oxalic acid. In the surface treatment, the electrodes 114 are dipped in an aqueous solution of oxalic acid, which is an etchant. The dipping time of the electrodes 114 in an aqueous solution of oxalic acid is, for example, about 50 seconds. The surface-treated electrodes 114 are washed with pure water or other liquids. In other words, the treatment time during which the electrodes 114 are dipped in the etchant in this embodiment is slightly shorter than the treatment time (e.g., 60 seconds) in Embodiment 1. Specifically, the treatment time in this embodiment is, for example, about 10 seconds shorter than the treatment time in Embodiment 1. Referring to FIG. 16, multiple grooves 117 are formed on the surface of the surface-treated electrodes 114. The formed grooves 117 are arranged on the entire surface of the electrodes 114 in a mesh manner, and the grooves 117 more assuredly have a depth equal to the thickness of the electrodes 114 (see FIG. 14 to FIG. 15).

The reasons why the depth of the grooves 117 in this embodiment is larger than that in Embodiment 1 although the treatment time during which the electrodes 114 are dipped in the etchant in this embodiment is shorter than the treatment time in Embodiment 1 will be described. A first reason is that an aqueous solution of oxalic acid is used as an etchant in this embodiment. Another reason is that, since the treatment time in the annealing treatment in this embodiment is shorter than that in Embodiment 1, the degree of crystallization of ITO contained in the electrodes 114 in this embodiment is lower than that in Embodiment 1, which increases the etching rate with the etchant in this embodiment. In this embodiment, the treatment time in the annealing treatment and the treatment time in the surface treatment are both shorter than those in Embodiment 1, so that the time it takes to produce the cell culture device 110 can be shorten.

In the cell culture device 110 of this embodiment, the grooves 117 have a depth equal to the thickness of the electrodes 114, and the electrodes 114 each include the island portions 18 divided by the grooves 117, as described above. The wires 112 are partially exposed through the grooves 117 when the electrodes 114 each include the island portions 18. The cells CE cultured on the surface of the electrodes 114 can be grown and spread out in a plane along the grooves 117 separating the island portions 18.

The method for producing the cell culture device 110 according to this embodiment includes forming the grooves 117 by etching the surface of the electrodes 114 by supplying an etchant containing oxalic acid to the electrodes 114. When the etchant containing oxalic acid is supplied to the electrodes 114, the surface of the electrodes 114 is etched to form the grooves 117. The grooves 117 thus formed each have a depth equal to the thickness of the electrodes 114. The electrodes 114 accordingly include the island portions 18 divided by the grooves 117. The wires 112 are partially exposed through the grooves 117 when the electrodes 114 each include the island portions 18. The cells CE cultured on the surface of the electrodes 114 can be grown and spread out in a plane along the grooves 117 separating the island portions 18.

### Other Embodiments

The technique disclosed in this specification is not limited to the embodiments described in the above description and drawings, and for example, the following embodiments are also included in the technical scope of the present disclosure.

(1) The treatment time and the treatment temperature in the annealing treatment and the surface treatment of the electrodes 14 and 114 in the production method can be appropriately changed from the time and the temperature described in Embodiments 1 and 2.

(2) The etchant used in the surface treatment of the electrodes 14 and 114 in the production method can be appropriately changed from an aqueous solution of iron(III) chloride and an aqueous solution of oxalic acid. For example, a mixed solution (aqua regia) of hydrochloric acid and nitric acid can also be used as an etchant.

(3) The groove width, the depth, the path in plan view, and other features of the grooves 17 and 117 can be appropriately changed from those described in Embodiments 1 and 2 and illustrated in the figures. The groove width of the grooves 17 and 117 may be greater than 10 nm and less than 10 The groove width of the grooves 17 and 117 may be equal to or less than 1/10 of a width of the contact holes 13A and 113A in the X-axis direction and the Y-axis direction.

(4) The planar shape, the size and arrangement spacing in plan view, and other features of the island portions 18 in Embodiment 2 can be appropriately changed from those described in Embodiment 2 and illustrated in the figures.

(5) The planar shape and the size in plan view of the first end portions 12A of the wires 12 and 112 can be appropriately changed from those illustrated in the figures. For example, the first end portions 12A may have a circular shape or other shapes.

(6) The planar shape and the size in plan view of the electrodes 14 and 114 can be appropriately changed from those illustrated in the figures. For example, the electrodes 14 and 114 may have a circular shape or other shapes.

(7) A specific range of the first main surfaces 11A and 111A of the substrates 11 and 111 in which the insulating films 13 and 113 are formed can be appropriately changed from that illustrated in the figures.

(8) A specific planar shape and a specific size in plan view of the contact holes 13A and 113A in the insulating films 13 and 113 can be appropriately changed from those described in Embodiments 1 and 2 and illustrated in the figures. For example, the planar shape of the contact holes 13A and 113A may be circular or other shapes.

(9) Specific numbers of the wires 12 and 112 and the electrodes 14 and 114 can be appropriately changed from those illustrated in the figures. For example, the number of the wires 12 or 112 may be 1, and the number of the electrodes 14 or 114 may be 1.

(10) A specific number of the wells 15 can be appropriately changed from that illustrated in the figures. For example, the number of the wells 15 may be two or more.

(11) A specific planar shape of the well 15 can be appropriately changed from that illustrated in the figures. For example, the planar shape of the well 15 may have a rectangular ring (frame) shape or other shapes.

(12) The materials used for the substrates 11 and 111, the wires 12 and 112, the insulating films 13 and 113, and the electrodes 14 and 114 can be appropriately changed from the materials described in Embodiments 1 and 2.

(13) The substrates 11 and 111, the insulating films 13 and 113, and the electrodes 14 and 114 may be made of translucent or opaque materials (e.g., light-shielding materials), in addition to transparent materials.

(14) The wires 12 and 112 may be made of a transparent or translucent material, in addition to a light-shielding material.

(15) The cell culture devices 10 and 110 may additionally include a processor that processes signals associated with action potentials acquired via the electrodes 14, 114, a display that shows analysis results, or other units. The processor can be composed of, for example, a microcomputer and may run an analysis program for analyzing signal data acquired from the electrodes 14 and 114 to, for example, count neural activity (spikes), detect bursts, and even analyze cellular networks in the long-term measurement of the action potentials for nerve cells. For muscle cells such as myocardium, the processor may, for example, measure extracellular potentials and analyze response potential data associated with various reactions during contraction and relaxation of the heart muscle.

The present disclosure contains subject matter related to that disclosed in Japanese Priority Patent Application JP 2022-177028 filed in the Japan Patent Office on Nov. 4, 2022, the entire contents of which are hereby incorporated by reference.

It should be understood by those skilled in the art that various modifications, combinations, sub-combinations and alterations may occur depending on design requirements and other factors insofar as they are within the scope of the appended claims or the equivalents thereof.

What is claimed is:

1. A cell culture device comprising:
a substrate;
a wire disposed on a main surface of the substrate;
an insulating film disposed on the main surface of the substrate and having a part that overlaps the wire; and
an electrode disposed on the insulating film and having a part that overlaps the wire,
wherein the insulating film has a contact hole at a position overlapping the wire and the electrode, and
a plurality of grooves are formed on at least a surface of a part of the electrode that overlaps the contact hole.

2. The cell culture device according to claim 1,
wherein the substrate and the insulating film are each made of a light-transmissive insulating material,
the wire is made of a light-shielding conducting material, and
the electrode is made of a light-transmissive conducting material.

3. The cell culture device according to claim 1, wherein the plurality of grooves have a depth smaller than a thickness of the electrode.

4. The cell culture device according to claim 1,
wherein the plurality of grooves have a depth equal to a thickness of the electrode, and
the electrode includes a plurality of island portions divided by the plurality of grooves.

5. The cell culture device according to claim 1, wherein the plurality of grooves are formed all over a surface of the electrode.

6. The cell culture device according to claim 1, wherein the plurality of grooves communicate with each other.

7. The cell culture device according to claim 1, wherein the plurality of grooves are arranged on the surface of the electrode in a mesh manner.

8. The cell culture device according to claim 1, wherein a groove width of the plurality of grooves is greater than 10 nm and less than 10 μm.

9. The cell culture device according to claim 1, wherein a groove width of the plurality of grooves is equal to or less than 1/10 of a width of the contact hole.

10. A method for producing a cell culture device, the method comprising:
disposing a wire on a main surface of a substrate;
disposing, on the main surface of the substrate, an insulating film having a part that overlaps the wire;
forming a contact hole in the insulating film at a position overlapping the wire;
disposing, on the insulating film, an electrode having a part that overlaps the wire and the contact hole; and
forming a plurality of grooves on at least a surface of a part of the electrode that overlaps the contact hole.

11. The method for producing a cell culture device according to claim 10, wherein the plurality of grooves are formed by etching a surface of the electrode.

12. The method for producing a cell culture device according to claim 11, wherein the plurality of grooves are formed by etching the surface of the electrode by supplying an etchant containing iron(III) chloride to the electrode.

13. The method for producing a cell culture device according to claim 11, wherein the plurality of grooves are formed by etching the surface of the electrode by supplying an etchant containing oxalic acid to the electrode.
